(19) United States
(12) Patent Application Publication
(10) Pub. No.: US 2011/0251664 A1
(43) Pub. Date: Oct. 13, 2011

(54) Short Legged Bifurcated Stent Graft Distal Capture Element and Method

(75) Inventor: Maria Acosta De Acevedo, Rohnert Park, CA (US)

(73) Assignee: Medtronic Vascular, Inc., Santa Rosa, CA (US)

(21) Appl. No.: 12/756,921

(22) Filed: Apr. 8, 2010

(51) Int. Cl. A61F 2/84; A61F 2/82

(52) U.S. Cl. 623/1.11; 623/1.35

(57) ABSTRACT

A bifurcated stent graft section includes a plurality of loops formed from a serpentine support wire forming a stent at a distal end of one of the short legs of the bifurcated stent graft section. A delivery system includes a coil having a wire configured to pass through the loops and prevent disengagement of the loops from the delivery system without relative rotational movement between the coil and the loops. In a method for delivery the bifurcated stent graft section, the bifurcated stent graft section is advanced to the treatment site, a sheath is retracted to deploy the proximal end of the bifurcated stent graft section while the coil retains the distal end of one of the short legs of the bifurcated stent graft section. A second guidewire is threaded into the short leg of the bifurcated stent graft section which is been stabilized by being held by the coil of the delivery system. An extension leg graft is delivered over the second guidewire and deployed to be coupled to the other short leg of the bifurcated stent graft section while the coil retains the loops. The coil is rotated to release the loops and allow the bifurcated stent graft section to fully deploy. A second extension leg graft is delivered and coupled to the other leg of the bifurcated stent graft section to complete the construction of a bifurcated stent graft in situ.

### TECHNICAL FIELD

This invention relates generally to stent grafts, stent graft delivery systems, and methods for delivering and deploying stent grafts to a desired location.

### BACKGROUND

Endovascular aneurysmal exclusion is an evolving method for treating arterial aneurysmal disease. Aneurysmal disease causes the weakening and radial distention of a segment of an artery. This arterial distention results in the development of an aneurysm, i.e., a bulging at the affected arterial segment.

An aneurysm is at risk of rupture resulting in extravasation of blood into, for example, the peritoneal cavity or into tissue surrounding the diseased artery. The goal of endovascular aneurysmal exclusion is to exclude from the interior of the aneurysm, i.e. aneurysmal sac, all blood flow, thereby reducing the risk of aneurysm rupture requiring invasive surgical intervention.

One procedure developed to accomplish this goal entails providing an alternate conduit effectively internally lining the affected artery with a biocompatible graft material. The graft material is configured in a generally tubular shape spanning the aneurysm (intra-aneurysmal). Stents are generally attached to the graft material to couple the graft material to the artery, establishing a substantially fluid-tight seal above and below the distended aneurysmal segment at graft/artery interfaces.

Endoluminal stent grafts are positioned and deployed within the affected artery through insertion catheters by percutaneous procedures well know to those of skill in the art. Once deployed, an endoluminal stent graft provides an alternate conduit for blood flow and, at the same time, prevents the flow of blood into the aneurysmal sac. Endoluminal stent grafts provide a generally effective means to exclude blood flow from aneurysms.

Proper matching of the size of a stent graft to the blood vessel in which it is to be deployed is critical to the treatment of an aneurysm. A stent graft preferably extends longitudinally along the vessel axis beyond the weakened portion of the blood vessel to anchor securely in healthy tissue of the vessel wall. However, the cross-sectional (diametral) size and axial length of individual blood vessels and aneurysms in those vessels vary considerably between patients. Even within a patient, the cross-section and resilience of a vessel wall can vary considerably along its axial length, and the location and extent of the aneurysm will differ with different patients. Additionally, each particularly sized stent graft must be carefully constructed and handled, making it extremely costly to provide and maintain a large range of sizes of stent grafts required to properly match the size needs of the treatable patients.

One solution to the costly large number of stent graft sizes required to be used in bifurcated regions such as an abdominal aortic aneurysm, is to provide modular stent grafts to construct a bifurcated stent graft assembly in vivo. For example, a primary bifurcated stent graft section may include a main body and two relatively short legs that are disposed in the primary vessel and do not extend into the branch vessels. After the primary bifurcated stent graft section is deployed in the primary vessel, two extension leg grafts are delivered and coupled to the short legs, and each extend into a respective branch vessel. Such modular stent graft assemblies allow for a smaller number and variety of sizes of the primary bifurcated stent graft section and extension leg grafts to be matched to the sizing needed by a particular patient and reduces the number of stent graft sizes required. However, with the primary bifurcated stent graft section deployed, control of the short legs may be difficult and threading a guidewire into a short leg and delivering the extension leg graft into the short leg of the bifurcated stent graft section may be complicated, difficult, and time consuming. Accordingly, a device and method for controlling a portion of a bifurcated stent graft after deployment is desirable.

### SUMMARY OF THE INVENTION

A bifurcated stent graft includes a plurality of loops formed from a serpentine shaped support at a distal end of one of its short legs. A delivery system includes a retention coil having a helically wound wire configured to pass through the loops and prevent disengagement of the loops from the delivery system without relative rotational movement between the retention coil and the loops. Rotating the coil in one direction causes the wire of the coil to capture the loops and hold the loops engaged with the delivery system. Rotating the coil in the opposition direction causes the wire of the coil to slip out of the loops to release the loops from the delivery system, allowing the short leg to which it is connected to expand and releases the short leg of the bifurcated stent graft section from the delivery system allowing the delivery system to be withdrawn from the patient.

In a method for delivery the bifurcated stent graft assembly, the primary bifurcated stent graft section is advanced to the treatment site, a sheath is retracted to deploy the primary bifurcated stent graft section while the coil retains the distal end of one of the legs of the primary bifurcated stent graft section. A first extension leg graft is delivered to and coupled to the contralateral short leg of the bifurcated primary bifurcated stent graft section while the coil retains the loops. The coil is then rotated to release the loops and allow the primary bifurcated stent graft section to fully deploy. A second extension leg graft may be delivered to and coupled to the second short leg of the bifurcated primary bifurcated stent graft section originally retained by the coil.

### BRIEF DESCRIPTION OF THE DRAWINGS

Embodiments will be further explained with reference to the appended Figures, wherein like structure is referred to by like numerals throughout the several views.

FIG. 1 illustrates a schematic plan view of a delivery system in accordance with the present invention.

FIG. 2 illustrates a close up view of a proximal portion of the delivery system of FIG. 1.

FIG. 3 illustrates a close up see through view of a bifurcated stent graft section enclosed in an outer sheath in a compressed configuration ready for intraluminal insertion or delivery to a desired anatomic site.

FIG. 4 schematically illustrates in partial cut away a portion of the screw gear and

FIG. 5 illustrates a bifurcated stent graft section.

FIG. 6 illustrates a capture tube with a wire coil attached thereto.

FIG. 7 illustrates the coil of FIG. 6 adjacent a portion of the bifurcated stent graft section of FIG. 5.

FIG. 8 illustrates the coil of FIG. 6 engaging loops of the short leg of the bifurcated stent graft section of FIG. 5.

FIG. 9 schematically illustrates the delivery system of FIG. 1 with the cover partially retracted.

FIG. 10 illustrates the delivery system of FIG. 1 with the cover fully retracted and the coil retaining the loops at the end of one of the short legs of the bifurcated stent graft section.

FIG. 11 illustrates the delivery system of FIG. 1 with the cover retracted and the coil released from the short leg of the bifurcated stent graft section.

FIGS. 12-14 cross sectionally schematically illustrate the delivery system of FIG. 1 being delivered through the vasculature to the site of an abdominal aortic aneurysm.

FIG. 15 illustrates the delivery system of FIG. 1 at the site of abdominal aortic aneurysm with the cover partially retracted such that the proximal portion of the bifurcated stent graft section has expanded. FIG. 15A illustrates the rotational movement of the external slider to controllably retract the cover to release the proximal portion of the bifurcated stent graft section.

FIG. 16 illustrates the delivery system of FIG. 1 with the cover fully retracted and the coil holding the first short leg of the bifurcated stent graft section. FIG. 16A illustrates the movement of the external slider to further retract the cover to release the bifurcated stent graft section.

FIG. 17 schematically illustrates a second delivery system for delivering a first extension leg graft to be mated with the second leg of the bifurcated stent graft section of FIG. 5.

FIG. 18 illustrates that the outer sheath of the second delivery system retracted such that the first extension leg graft expands and is coupled to the second leg of the bifurcated stent graft section of FIG. 5.

FIG. 19 illustrates the coil of the first delivery system having been rotated such as to release the first leg of the bifurcated stent graft section from the coil.

FIG. 20 illustrates movement of the inner tube such that the tip is retracted to the cover such that the delivery system can be removed.

FIG. 21 illustrates a third delivery system for delivering a second extension leg graft to be mated with the first leg of the bifurcated stent graft section of FIG. 3.

FIG. 22 illustrates the outer sheath of the third delivery system retracted such that the second extension leg graft has self expanded and is coupled to the first leg of the bifurcated stent graft section of FIG. 5.

FIG. 23 illustrates the bifurcated stent graft section of FIG. 5 and two extension leg grafts implanted at the site of an abdominal aortic aneurysm.

### DETAILED DESCRIPTION

With reference to the accompanying figures, wherein like components are labeled with like numerals throughout the figures, an illustrative delivery system and method of the delivering an endoluminal bifurcated stent graft section is disclosed, taught and suggested by the multiple embodiments.

Unless otherwise indicated, with respect to stent grafts described herein such as stent graft 200, the terms “distal” and “proximal” are used in the following description with respect to a position or direction relative to the heart. “Distal” and “distally” are positions distant from or in a direction away from the heart by way of blood flow path, and “proximal” and “proximally” are positions near or in a direction closest to the heart by way of blood flow path. With respect to delivery systems described herein, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician.

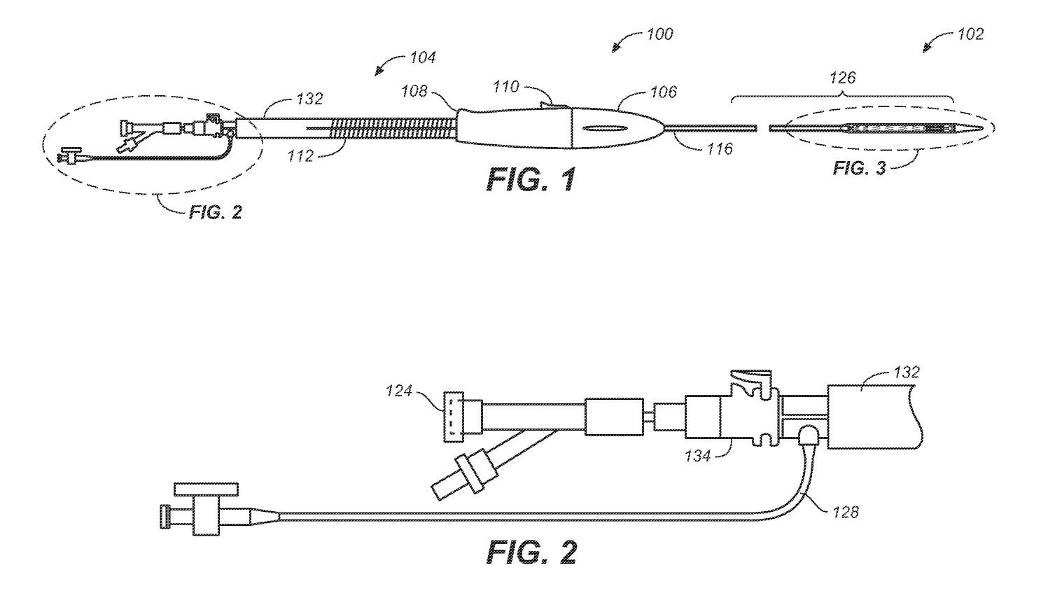

Referring now to the FIGS. 1-4, wherein components are labeled with like numerals throughout the several figures, an embodiment of a delivery system 100 and bifurcated stent graft section 200 is shown. Delivery system 100 includes a distal end 102 and a proximal end 104. Distal end 102 is preferably used to load and deliver bifurcated stent graft section 200. Proximal end 104 includes components such as those found conventionally in catheter delivery systems.

The components of the proximal end 104 of the delivery system 100 may preferably include those shown in FIGS. 1 and 2, although additional and/or alternative components are also contemplated. In particular, proximal end 104 of delivery system 100 includes a Touhy Borst adaptor 124, a quick disconnect 134, a sideport extension 128, a rear grip 132, a screw gear 112, an external slider 108, a front grip 106, and a strain relief 116. One or more hemostatic valves may be provided in front grip 106, for example, as described in U.S. Published Patent Application Publication No. 2006/0229561, commonly assigned with the present applications, which is incorporated herein by reference in its entirety. The delivery system 100 as described is generally similar to the Xcelerant Delivery System, sold by Medtronic, Inc., but may be any conventional therapy delivery system, with modifications noted in detail below. Delivery system 100 is generally a single use, disposable device with the bifurcated stent graft section 200 mounted on within distal end 102 of the delivery system 100.

Delivery system 100 is coaxially configured and includes an inner tube 114 providing a guidewire lumen therethrough, and is connected to a guidewire lumen inlet at the proximal end of inner tube 114. Inner tube 114 preferably extends along the entire length of delivery system 100 to allow a guidewire to pass from the proximal end 104 and out through the distal end 102. Inner tube 114 may be slid or moved relative to other surrounding components of the system 100 by releasing the quick disconnect 134, holding and moving the proximal end of the inner tube 114 or the Touhy Borst adaptor 124. Delivery system 100 may be used with a guidewire, as described below, which may be, for example a 0.035 inch (0.089 cm) extra stiff guidewire as manufactured by Amplatzer, Golden Valley, Minn., U.S.A. The guidewire may be used to guide the delivery system 100 to its desired implant location. Other guidewires may also be used.

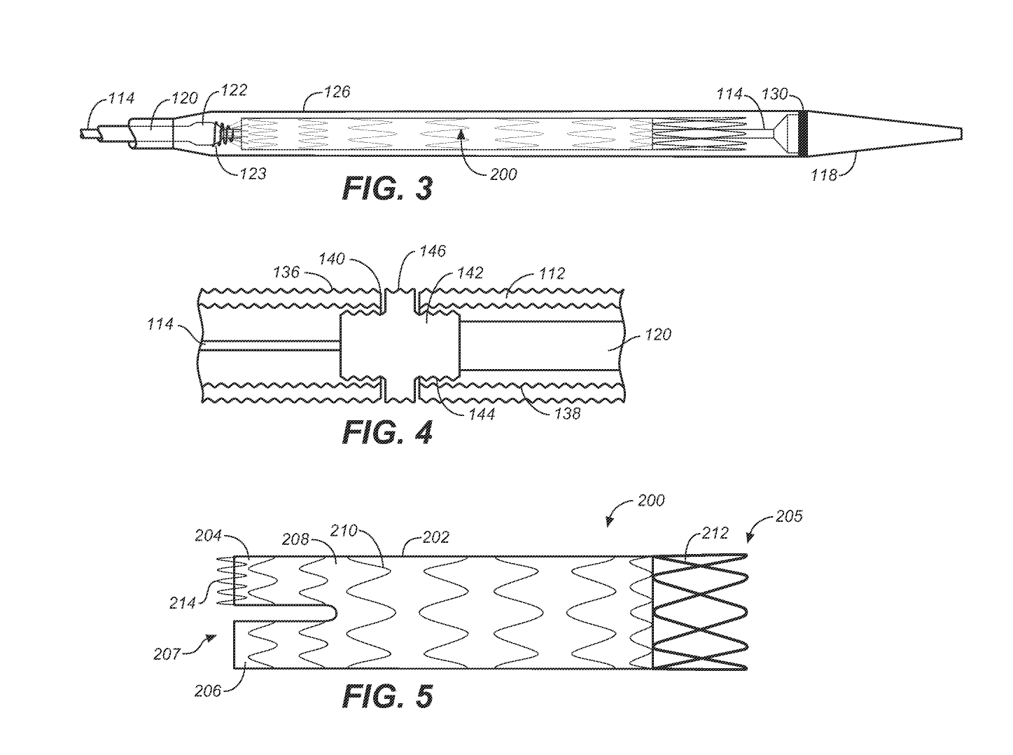

FIG. 3 shows the distal end 102 of the system 100 in a predeployment configuration with bifurcated stent graft section 200 loaded therein. Attached to the inner tube 114 is a tapered tip 118, which serves to ease the passage of the delivery system 100 through the vasculature. An outer tube or cover 126 is disposed around bifurcated stent graft section 200. Cover 126 in the delivery configuration extends from tip 118 proximally and is coupled to external slider 108. Cover 126 moves longitudinally relative to inner shaft 114 and bifurcated stent graft section 200 as external slider 108 is moved relative to screw gear 112 to release bifurcated stent graft section 200 from the delivery system 100, as explained in more detail below. A radiopaque marker 130 may be placed at a distal end of cover 126 in order to aid in visualization of cover 126 during delivery and deployment of bifurcated stent graft section 200. Cover 126 is preferably made of a low friction and flexible material, such as polytetrafluoroethylene (PTFE), polyurethane, silicone, or polyethylene, and is sized and shaped to house other distal end 102 components of the delivery system 100.

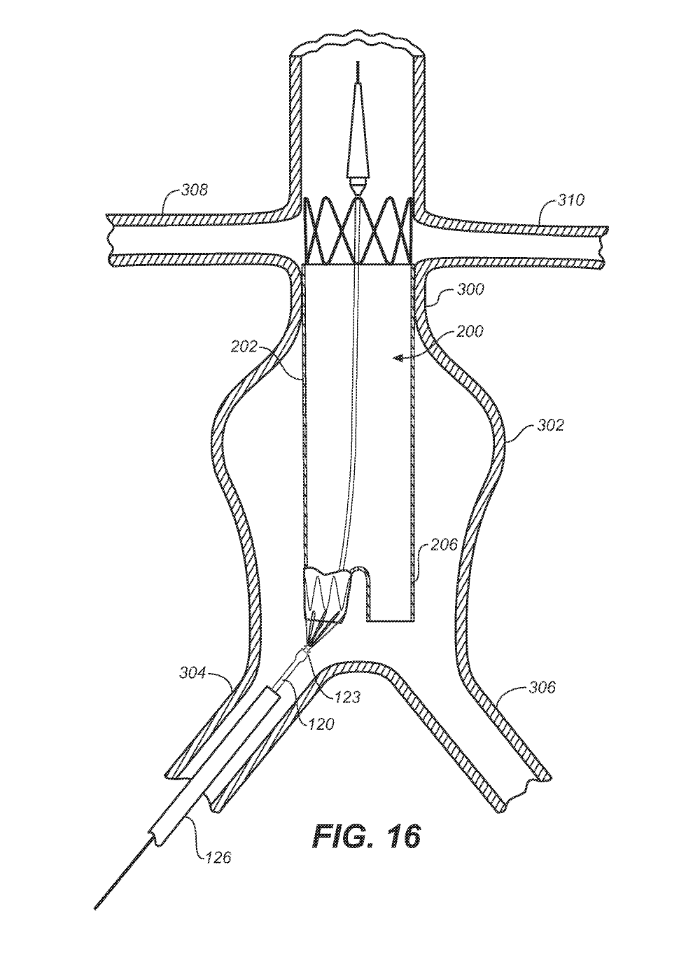

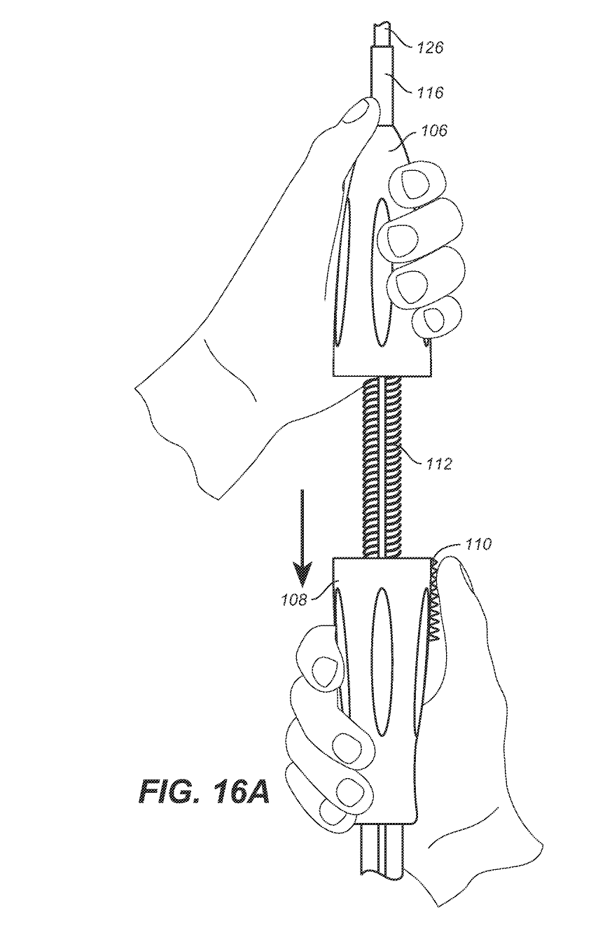

Also shown is a middle member or device capture tube 120, including a device capture element 122 holding or integral with a coil 123. The coil 123 retains the loop at the end of the short leg of the bifurcated stent graft section 200 until deployed, as described in more detail below. Device capture tube 120 includes a centrally located lumen surrounding inner tube 114. Device capture tube 120 is able to rotate relative to inner tube 114. Device capture tube 120 is similar to a middle member of Medtronic, Inc.'s Xcelerant Delivery System, with differences noted herein. The device capture element 122 at the distal end of the device capture tube 120 is a generally bulbous shape, although it may be any suitable shape (and may fully fill the circular (tubular) cross sectional space behind the compressed stent graft). In addition to holding coil 123, capture element 122 acts as a stent stop to prevent bifurcated stent graft section 200 from sliding back as cover 126 is retracted. Rotation of device capture tube 120 is utilized to engage or disengage capture element 122 and coil 123 from the loops of a short leg of bifurcated stent graft section 200. As shown in FIG. 4, device capture tube 120 extends to the proximal end 104 of system 100 within screw gear 112. A spindle 142 is attached to or integral with a proximal end of device capture tube 120 such that device capture tube 120 rotates as spindle 142 is rotated. An outer surface of spindle 142 includes circular ribs 144 that mate with circular grooves 138 on an inner surface of screw gear 112. The ribs/grooves 144/138 are for rotational movement only such that spindle 142 does not translate axially relative to screw gear 112 when spindle 142 is rotated. Screw gear 112 includes a window (region of longitudinal separation) 140 through its wall for access to spindle 142. Window(s) 140 may extend around the entire circumference of screw gear 112, or only around as much as is required to access the spindle with an outside manipulation to rotate coil 123 to engage/disengage coil 123 to/from one of the legs of the bifurcated stent graft section 200, as described in more detail below. Spindle 142 may include an extension 146 to extend through window 140 to an outside surface of screw gear 112. Spindle 142 may be located within screw gear 112 at a location such that when external slider 108 is retracted proximally to retract cover 126, as shown in FIG. 16A, spindle 142 would be located longitudinally between external slider 108 and front grip 106. As described in more detail below, coil 123 provides a means to hold a leg of bifurcated stent graft section 200 during delivery to and deployment of bifurcated stent graft section 200 at a desired location.

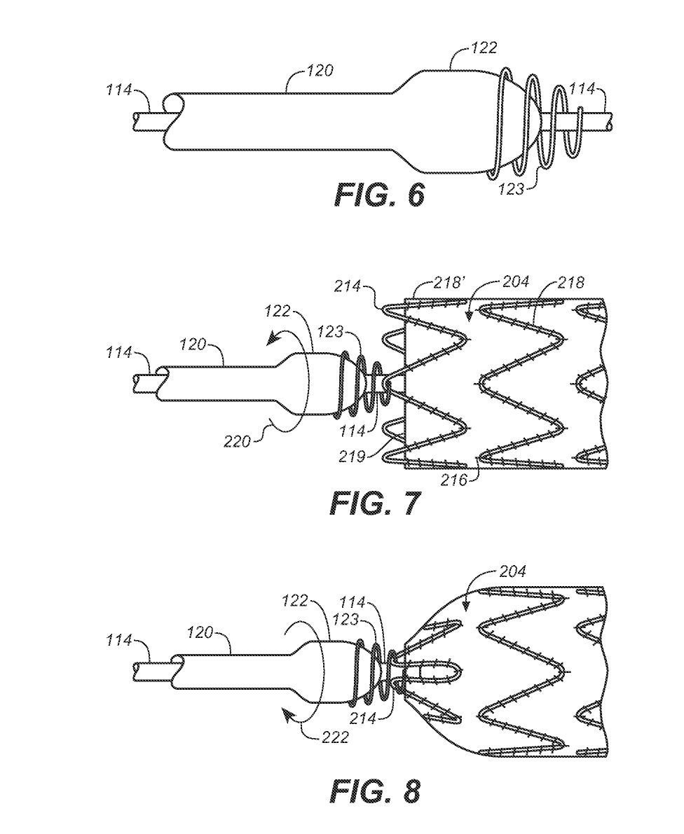

FIG. 5 illustrates the exemplary bifurcated stent graft section 200 to be delivered by delivery system 100. Bifurcated stent graft section 200 includes a main body 202, a first short leg 204, and a second short leg 206. Bifurcated stent graft section 200 includes a proximal portion 205 and a distal portion 207, with the first and second short legs 204, 206 disposed at the distal portion 207. Bifurcated stent graft section 200 may be any conventional stent graft known to those of skill in the art and is compressible to be inserted via catheter and released to expand to fit a desired body lumen, such as the aorta, for example. Bifurcated stent graft section 200 includes a circumferential or perimeter support or frame 210 attached to graft material or fabric 208. Support 210 is preferably formed and made from a framework that comprises a wire or plurality of wires 218 made of a shape-memory material (e.g., nitinol). Support 210 is attached to graft material 208 by stitching 216, for example, as shown in FIGS. 5 and 6.

The rings or loops of wires 218 of support 210 are shaped and aligned such that the wires 218 are aligned generally coaxially, so that a central lumen runs along the length of main body 202. The wires 218 preferably form a path that include a series of sinusoidal bends as they form a ring around the circumference of the graft, which allow for the compression and expansion of the support 210. The wires 218 may be attached separately to the graft material 208 or may be part of a framework. At the proximal portion 205 of the bifurcated stent graft section 200, a (bare) spring stent 212 extends beyond the proximal end of the graft material 208, as shown in FIG. 3.

The graft material 208 used for bifurcated stent graft section 200 may be a polyester knit, for example, or may be ultra high molecular weight polyethylene (UHMWPE), cotton, ePTFE, or the like. The graft material 208 should be biocompatible and may include a number of different fabrics in different areas of the bifurcated stent graft section 200 and/or in layers.

The wire 218′ of the support 210 at the distal end of at least one of the short legs of the bifurcated stent graft section 200, in this embodiment the first short leg 204, extend distally beyond a distal end 219 of the graft material 208, forming loops 214 as shown in FIGS. 5, 7, and 8. In the embodiment of bifurcated stent graft section 200 shown in FIG. 5, first and second short legs 204, 206 are approximately the same length. However, one skilled in the art would recognize that the legs may be constructed with one being longer than the other. In this particular embodiment, both first and second short legs 204, 206 are relatively short so as not to extend into the branch vessels of the bifurcated vessels, as explained in more detail below. The loops could also be formed by individual unconnected structures or by less rigid structures as stitches with suture like material.

FIG. 6 shows a distal portion of the device capture tube 120, including capture element 122 and coil 123. As shown, capture element 122 is integral with device capture tube 120. Coil 123 (which is constructed of a decreasing radius helical wire shape) is attached to capture element 122 by adhesive, fusion, or other attachment elements know to those skilled in the art. Coil 123 may also be integral with capture element 122 and/or device capture tube 120. Coil 123 is sized and configured to capture loops 214 of bifurcated stent graft section 200, as shown in FIGS. 7 and 8.

FIG. 7 shows capture element 122 with coil 123 positioned adjacent to a loop 214 at the distal end of first short leg 204 of bifurcated stent graft section 200. Once the ends of the loops 214 are compressed to be positioned close to each other and to the radial dimension of the coil 123, rotating device capture tube 120 in the direction of arrow 220 and directing the end of the wire of the coil 123 through the opening in the loop 214 causes coil 123 to rotate in the same direction, thereby capturing loops 214 of first short leg 204 of bifurcated stent graft section 200 and causing loops 214 to gather towards each other, as shown in FIG. 8. The loops 214 can be captured consecutively around the circumference or by capturing alternate loops (peaks) around the circumference. The wire capture path can be consistent, i.e., threading the wire end inside-out on each loop or can be outside-in or alternate between inside-out and outside-in. The configuration of FIG. 8, with loops 214 gathered in coil 123, permits the user to control the position of the first short leg 204 of bifurcated stent graft section 200 from the proximal end of the delivery system 100, such as by second valve 110. Rotating device capture tube 120 in the direction shown by arrow 222 in FIG. 8 causes the wire of the coil 123 to unwind from the loops and releases loops 214 from coil 123 such that first short leg 204 is released and self expands to the configuration shown in FIG. 7. Capture tube 120 may then be withdrawn proximally, leaving bifurcated stent graft section 200 in the desired location, as explained in more detail below.

With the bifurcated stent graft section 200 loaded in the delivery system 100 as shown in FIG. 3, a user uses the delivery system to percutaneously deliver the bifurcated stent graft section to a placement site. The proximal end 104 of the delivery system 100 remains outside of the patient, and the distal end 102 is inserted into the patient. The proximal end 104 provides features which allow manipulation of delivery system components including means for remotely controlling the distal end 102 of the system 100. The bifurcated stent graft section 200 is coupled to the delivery system 100 by causing coil 123 to be turned as the stent graft end loops 214 are compressed so as to gather and engage capture loops 214 of bifurcated stent graft section 200. The bifurcated stent graft section 200 is loaded in the delivery system 100 in a radially compressed configuration by methods known to those skilled in the art.

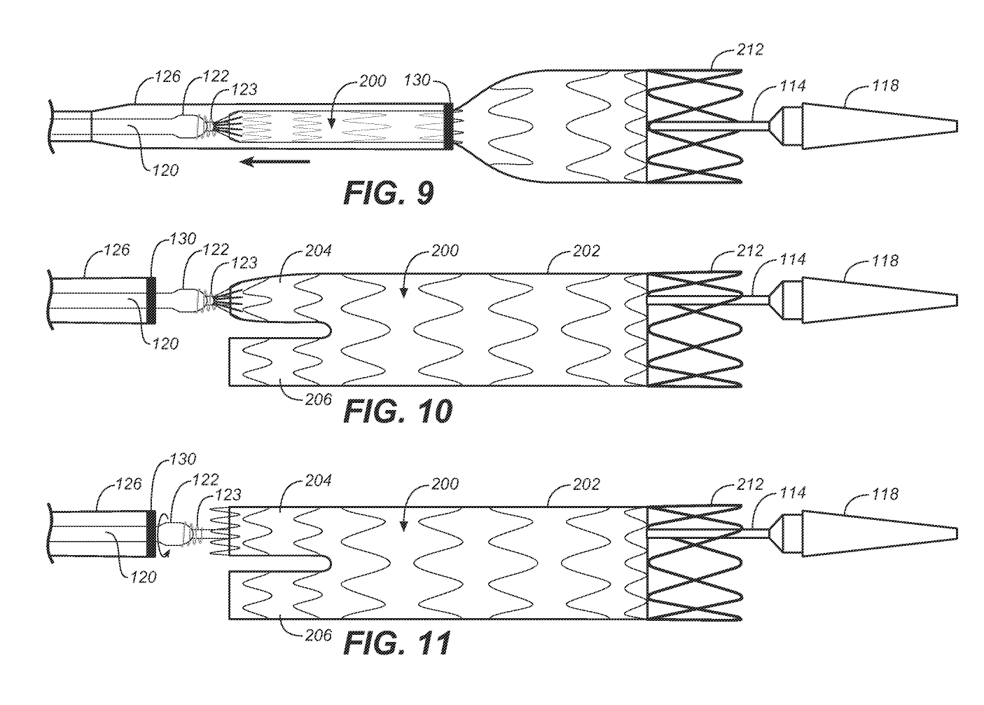

To deploy or release the bifurcated stent graft section 200 once it has been positioned at its desired location in the vasculature, the cover 126 is retracted proximally by proximal movement of external slider 108. As shown in FIG. 9, the radially compressed bifurcated stent graft section 200 is shown partially released. In FIG. 10, the cover 126 has been fully retracted until the cover 126 no longer surrounds the bifurcated stent graft section 200, while loops 214 of first short leg 204 of bifurcated stent graft section 200 are still attached to the coil 123. At this stage of deployment, second short leg 206 also has been fully released from cover 126. Until the loops 214 are released from the coil 123 by rotation of spindle 142, the distal portion of bifurcated stent graft portion is held in a stationary (stable) position by the attached delivery system. To release the bifurcated stent graft section 200, spindle 142 is rotated to rotate device capture tube 120 and coil 123, as shown in FIG. 8 such that rotation of the coil 123 releases loops 214 of first short leg 204, as shown in FIG. 11.

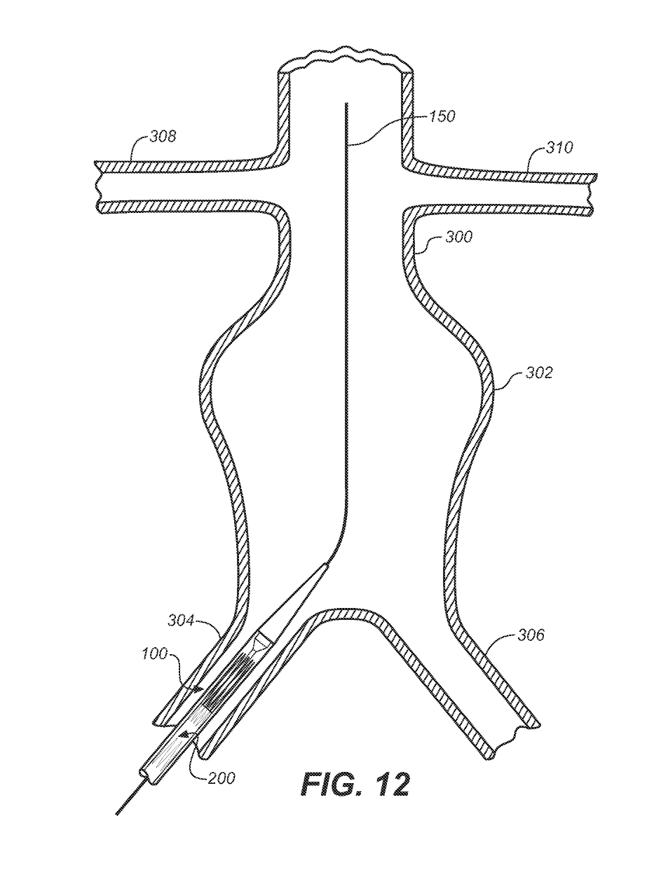

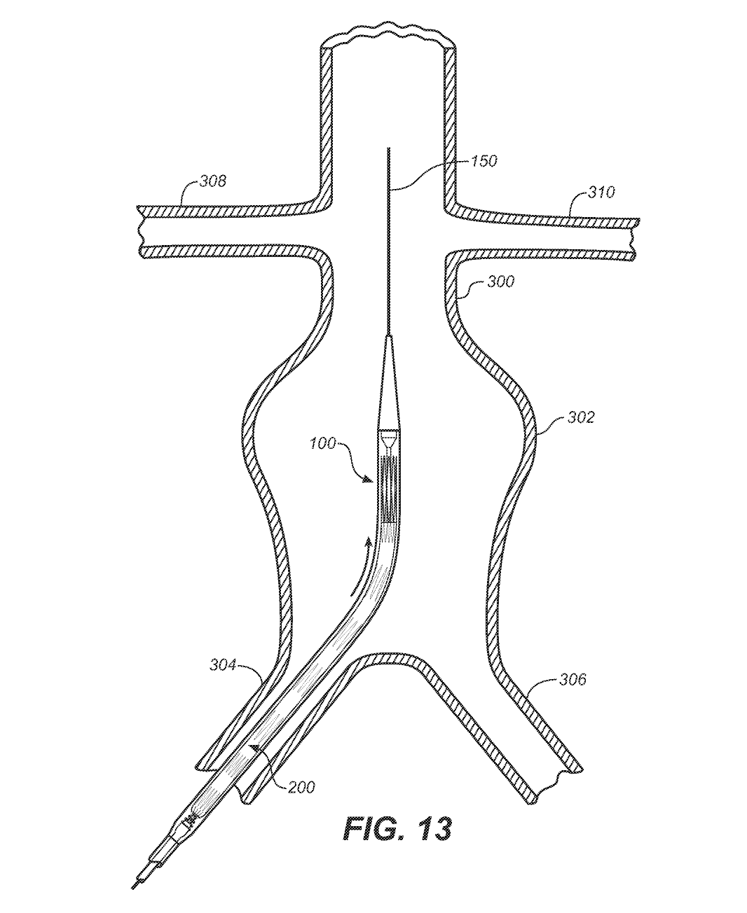

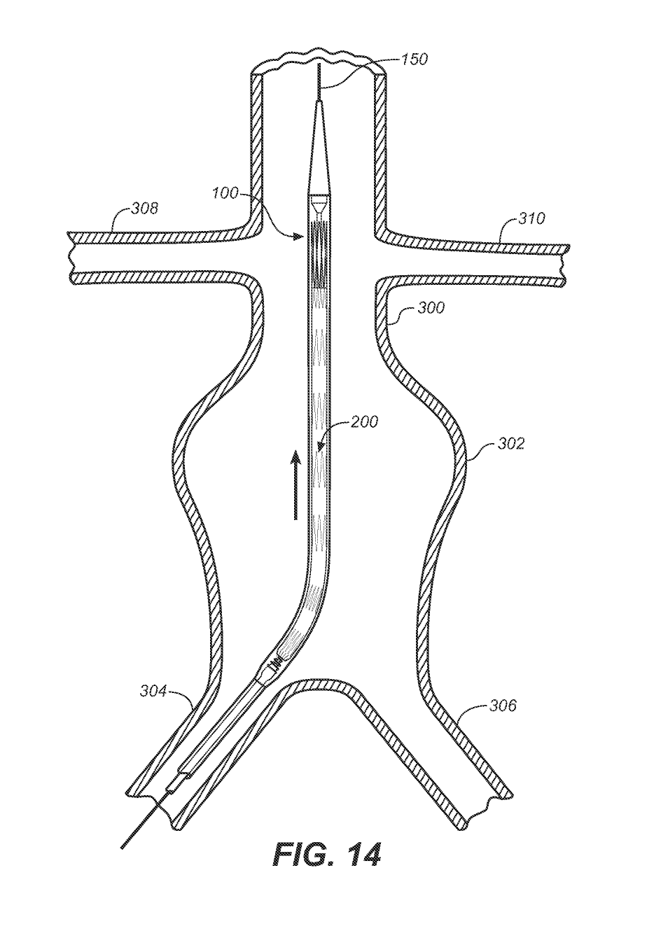

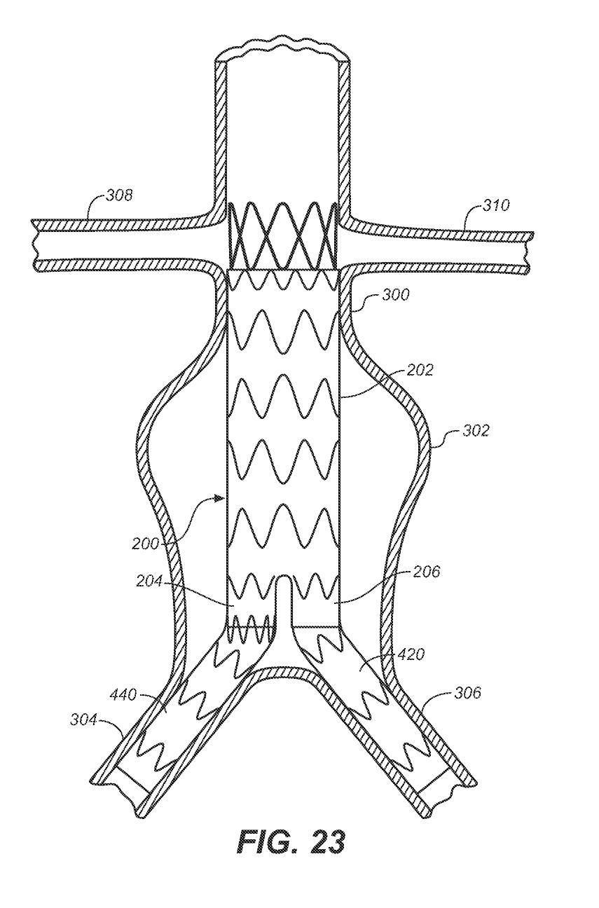

FIGS. 12-23 illustrate the steps of a method of delivering and deploying a bifurcated stent graft section to the site of an abdominal aortic aneurysm. FIG. 12 shows the abdominal aorta 300 with an aneurysm 302. Also shown are the right common iliac artery 304, the left common iliac artery 306, the right renal artery 308, and left renal artery 310. Referring to FIG. 12, a guidewire 150 is advanced through the vasculature and into the abdominal aorta 300. In the embodiment shown, guidewire 150 is advanced through the right common iliac artery 304, but it is contemplated that guidewire 150 could be advanced through the left common iliac artery 306, or by any other suitable pathway known to those skilled in the art. Delivery system 100, with bifurcated stent graft section 200 loaded therein, is advanced over guidewire 150 to the desired location in the abdominal aorta, as shown in FIGS. 12-14.

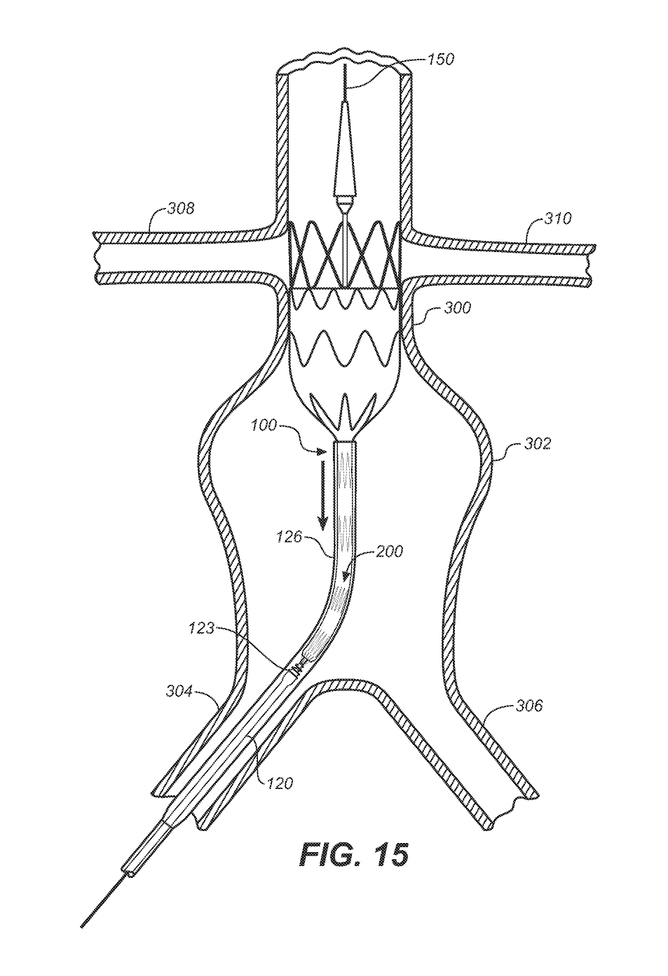

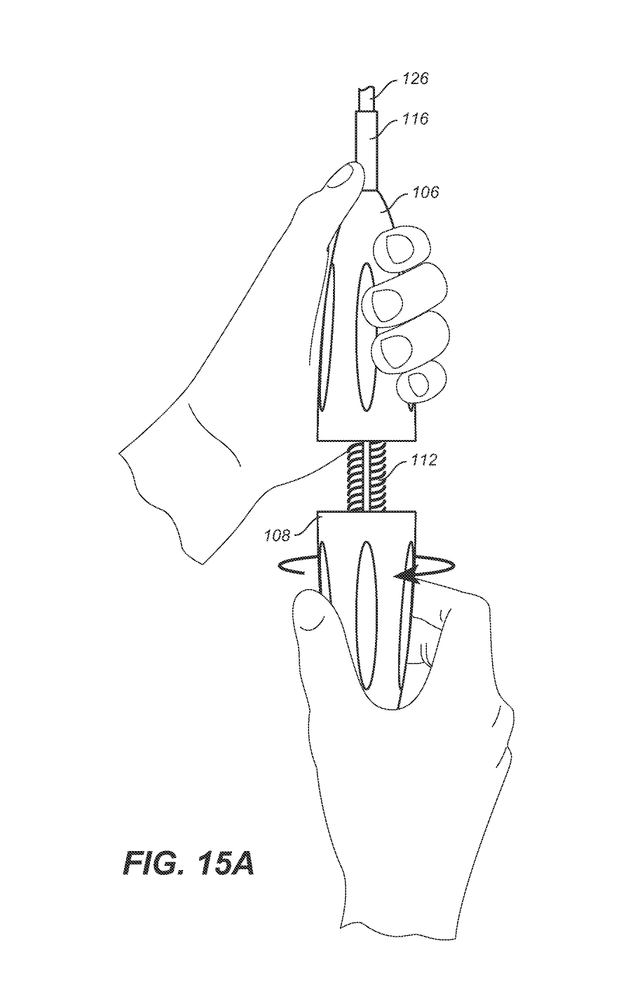

Once the delivery system is in the proper location, cover 126 is retracted by retracting external slider 108. External slider 108 may first be retracted by counter-clockwise rotational movement, as shown in FIG. 15A. This rotational movement provides a slower retraction of cover 108 for a controlled release of the proximal portion 205 of bifurcated stent graft section 200, as shown in FIG. 15. After the cover 126 has been retracted such that the (bare) spring stent 212 and the first support 210 coupled to the graft material 208 have fully expanded and opposed the aortic wall, the cover 126 is further retracted by further retracting external slider 108. This further retraction of external slider 108 may be done more quickly than the initial controlled retraction by pressing trigger 110 and sliding external slider 108, as shown in FIG. 16A, rather than rotating external slider 108. When outer sheath 126 has been retracted past the distal end of bifurcated stent graft section 200, as shown in FIG. 16, main body 202 and second short leg 206 of bifurcated stent graft section 200 have deployed and expanded. In the embodiment shown, second short leg 206 of the bifurcated stent graft section 200 is “above” the bifurcation in that second short leg 206 is disposed entirely in the abdominal aorta 300 and does not extend into the left common iliac artery 306. Similarly, first short leg 204 of bifurcated stent graft section 200 may be disposed entirely within the abdominal aorta 300 and not extend into the right common iliac artery 304. Coil 123 is holding loops 214 of first short leg 204 such that the position of the first short leg 204 can be controlled and visualized by device capture tube 120, which may be impregnated with radiopaque material to enhance visibility.

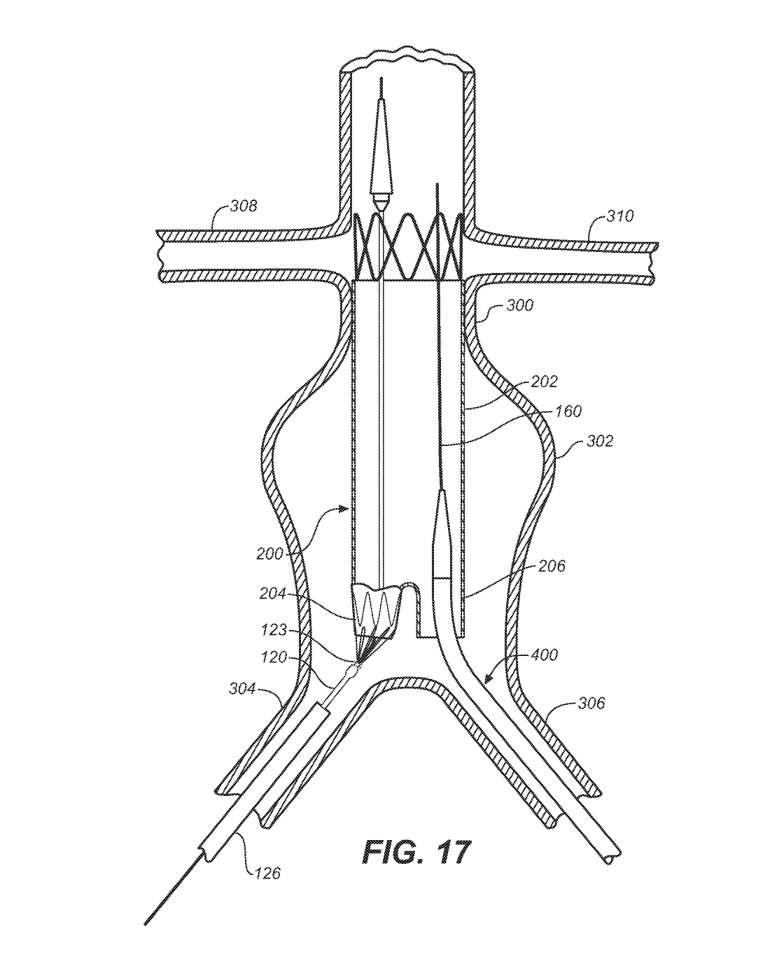

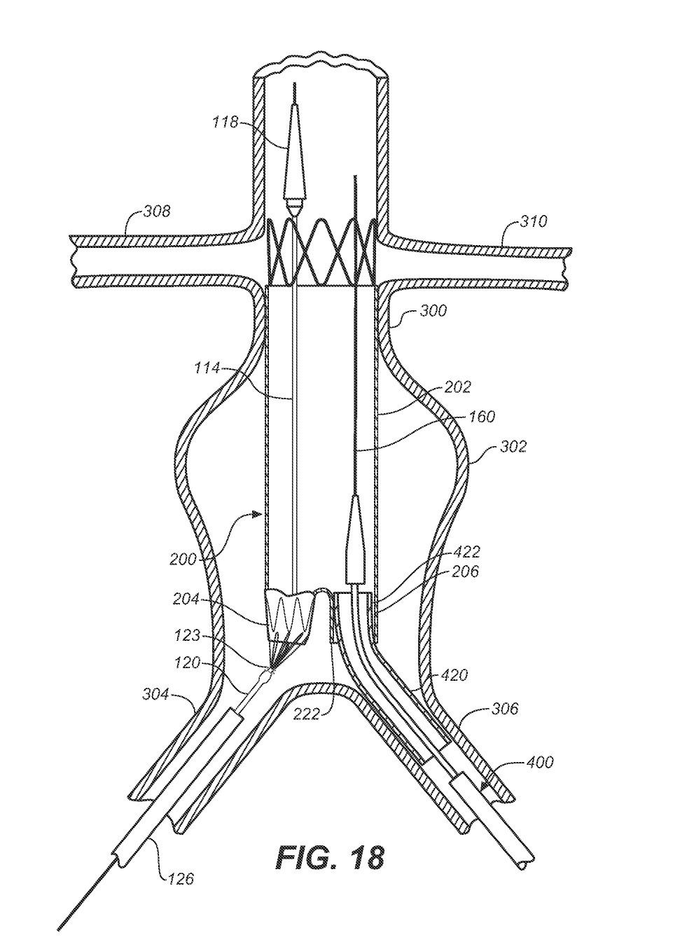

With coil 123 still holding first short leg 204, a second guidewire 160 is advanced in the contralateral limb with the object of threading the end of the guidewire into the opening (or lumen) at the end of the second short leg 206. Without the coil 123 holding the end of the first short leg 204, it and the distal end of the second short leg 206 could move and flap unpredictably as the pulsating flow of blood from the heart rushes through the bifurcated stent graft section 200 whose proximal end has been fixed within the aorta above the aneurysm. The presence of the coil 123 and the force it exerts on the end of the first short leg 204, provides spatial stability to the distal end of the first short leg 204 and transfers this stabilizing force through the distal portion of the bifurcated stent graft section to reduce the movement and unpredictability of the second short leg 206 during the time when an attempt is being made to thread the end of the second guidewire 160 into the opening at the end of the second short leg 206. Once the second guidewire 160 has been threaded into the second short leg 206 and further into and through the bifurcated stent graft section 200, a second delivery device 400 is advanced over the second guidewire 160 through the left common iliac artery 306 and into the abdominal aorta 300, as shown in FIG. 17. Second delivery device 400 is advanced to a point such that a portion of second short leg 206 overlaps with a portion of a first extension leg graft 420 compressed in the second delivery device 400. An outer sheath or cover of the second delivery device 400 is then retracted to release the first extension leg graft 420, as shown in FIG. 18. As also shown in FIG. 18, a distal portion 222 of second short leg 206 of bifurcated stent graft section 200 overlaps with a proximal portion 422 of the expanded first extension leg graft 420. This coupling of first extension leg graft 420 to second short leg 206 of bifurcated stent graft section 200 provides a continuous conduit through the abdominal aorta 300 and into the left common iliac artery 306 to bypass aneurysm 302. The position of the main body of the stent graft is now stable as it is fixed to the vessel wall at its proximal end by the proximal end of the bifurcated stent graft section 200 and then to the vessel wall at the distal end in the branch vessel (iliac artery) containing the distal end of the first extension graft leg 420. The overlapping connection between the bifurcated stent graft section second short leg 206 and the first extension leg graft 420 provides a tensile and compressive force connection thereby stabilizing the movement of the stent graft as compared to when both short legs of the bifurcated stent graft section are flapping unrestrained in the blood flow.

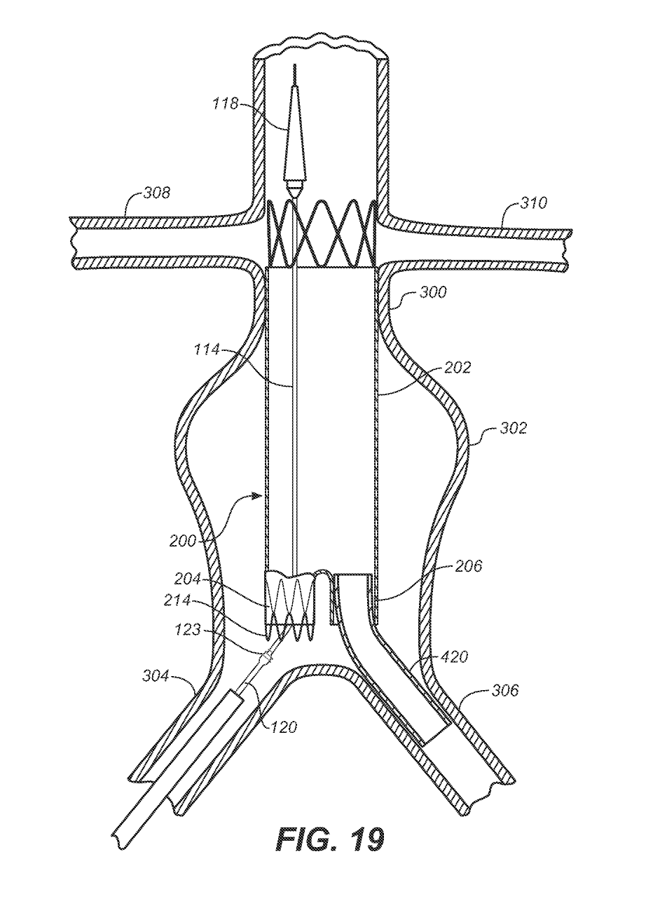

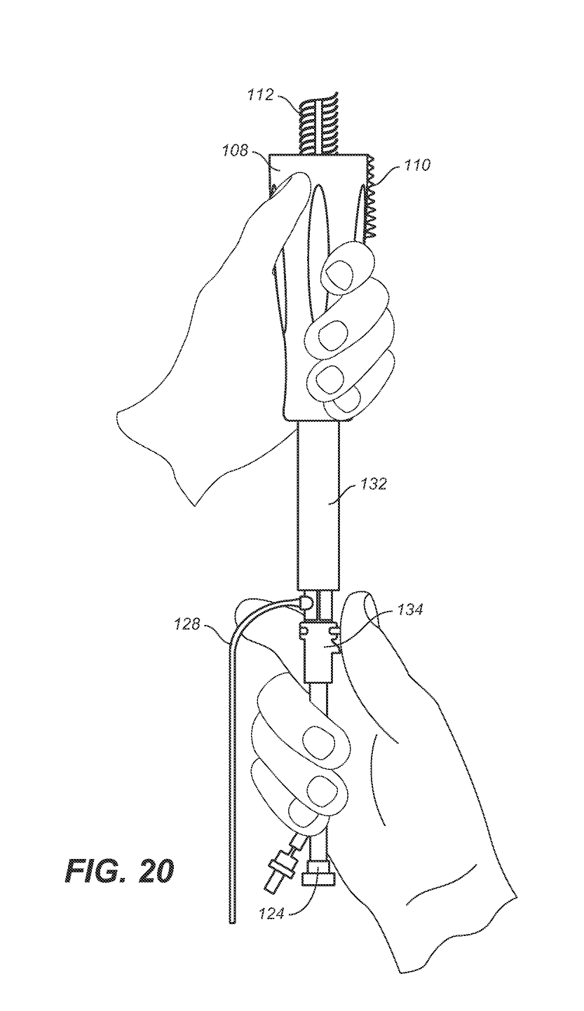

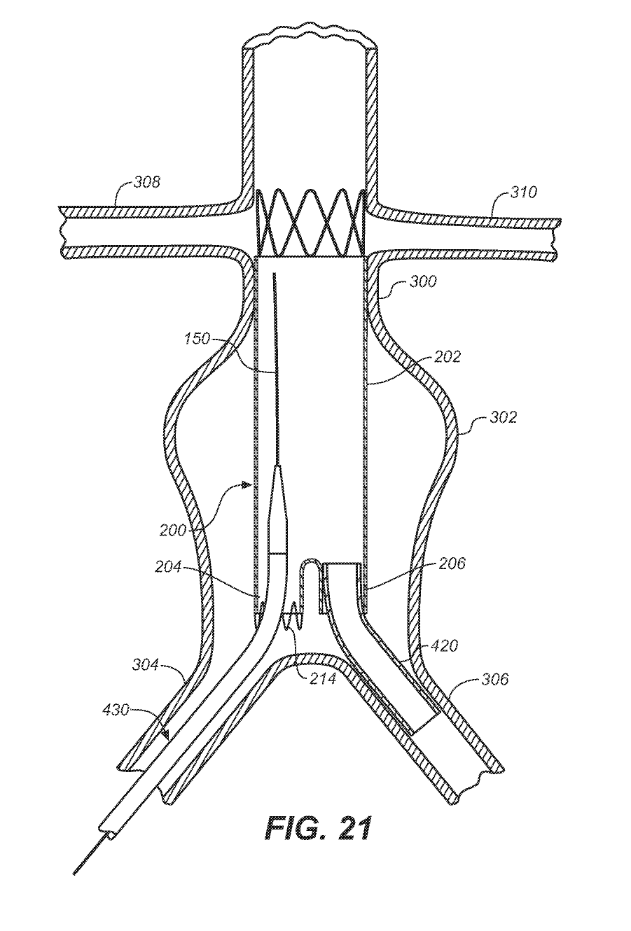

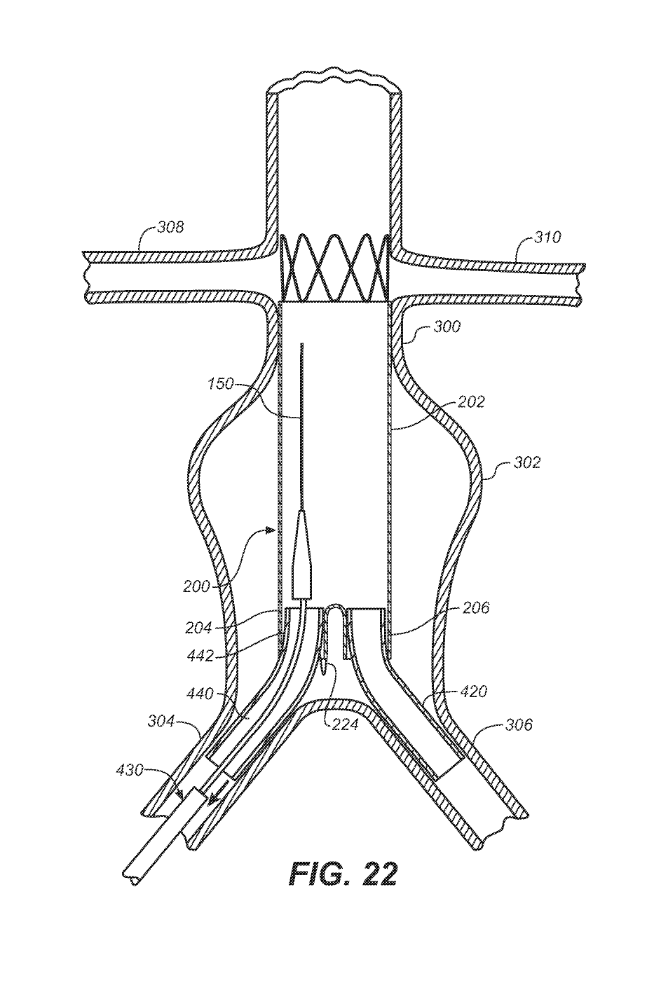

After first extension leg graft 420 is deployed, device capture tube 120 and coil 123 are rotated by rotating spindle 142 such that coil 123 releases loops 214 of first short leg 204, as shown in FIG. 19. Delivery system 100 can be withdrawn from the patient's body by pressing quick disconnect 134 to retract tip 118 into cover 126, as shown in FIG. 20, and then withdrawing the entire delivery system 100. A third delivery device 430 may be advanced over guidewire 150 through the right common iliac artery 304 and into the abdominal aorta 300, as shown in FIG. 21. Third delivery device 430 is advanced such that a portion of first short leg 204 overlaps with a portion of a second extension leg graft 440 disposed in third delivery device 430. An outer sheath or cover of the third delivery device 430 is then retracted to release the second extension leg graft 440, as shown in FIG. 22. As also shown in FIG. 22, a distal portion 224 of first short leg 204 of bifurcated stent graft section 200 overlaps with a proximal portion 442 of second extension leg graft 440. This coupling of second extension leg graft 440 to first short leg 204 of bifurcated stent graft section 200 provides a continuous conduit through the abdominal aorta 300 and into the right common iliac artery 304 to bypass aneurysm 302. The third delivery device 430 may then be withdrawn from the patient, leaving a bifurcated stent graft assembly in place, as shown in FIG. 22.

The foregoing detailed description and examples have been given for clarity of understanding only. No unnecessary limitations are to be understood therefrom. It will be apparent to those skilled in the art that many changes can be made in the embodiments described.

What is claimed is:

1. A method of delivering a bifurcated stent graft to a desired anatomic site, the method comprising the steps of:
advancing a delivery system intraluminally toward the anatomic site, wherein the delivery system includes:
a bifurcated stent graft section mounted therein, the bifurcated stent graft section including a main body and two legs extending from the main body, the bifurcated stent graft section having a graft material and a support coupled to the graft material, wherein a portion of said support coupled to a first leg of said two legs has a capture structure having a series of loops extending beyond a first end of the first leg opposite where the first leg is coupled to the main body;
an elongated capture tube having a proximal end, a distal end, and a coil coupled to the distal end and passing through said plurality of loops at the first end of the first leg of the bifurcated stent graft section to prevent disengagement of the loops from said delivery system, and
an elongated outer sheath, wherein the capture tube is rotatably disposed within a lumen of the outer sheath, the sheath enclosing the bifurcated stent graft section in a collapsed configuration for delivery to the desired anatomic site;
upon reaching the anatomic site, retracting the outer sheath to allow the bifurcated stent graft section to partially expand such that the main body and the second leg expand;
while maintaining the coil engaged with the plurality of loops at the first end of the first leg, advancing a second delivery system including a first extension leg graft disposed therein into the second leg of the bifurcated stent graft section;
deploying the first extension leg graft such that a proximal end of the first extension leg graft is coupled to a distal end of the second leg; and
rotating the capture tube to release the plurality of loops from the coil, thereby allowing the first end of the first leg to expand and releasing the bifurcated stent graft from the delivery system.

2. The method of claim 1, wherein the first extension leg graft is deployed by retracting an outer sheath of the second delivery system.

3. The method of claim 1, further comprising the step of advancing a third delivery system including a second extension leg graft disposed therein into the first leg of the bifurcated stent graft section and deploying the second extension leg graft such that a proximal end of the second extension leg graft is coupled to the first end of the first leg of the bifurcated bifurcated stent graft section.

4. The method of claim 3, wherein the second extension leg graft is deployed by retracting an outer sheath of the third delivery system.

5. The method of claim 1, wherein the desired anatomic site is the abdominal aorta of a patient, and wherein the bifurcated stent graft section is deployed in the abdominal aorta and the first extension leg graft is deployed in one of the left iliac artery and the right iliac artery.

6. The method of claim 3, wherein the desired anatomic site is the abdominal aorta of a patient, and wherein the bifurcated stent graft section is deployed in the abdominal aorta, the first extension leg graft is deployed in one of the left iliac artery and the right iliac artery, and the second extension leg graft is deployed in the other of the left iliac artery and the right iliac artery.

7. The method of claim 6, wherein the first leg and the second leg of the bifurcated stent graft section are disposed in the abdominal aorta and do not extend into the left iliac artery or the right iliac artery.

8. The method of claim 1, wherein the delivery system further comprises a rotatable element disposed at a proximal portion of the delivery system, wherein the rotatable element is coupled to capture tube, and wherein the step of rotating the capture tube comprises rotating the rotating element.

9. The method of claim 1, wherein the portion of the support structure forming the loops of the capture structure has a serpentine shape along a circumference of the first leg at the first end of the first leg, the support extending beyond an end of the graft material at the first end of the first leg such that the serpentine shape forms the plurality of loops extending from the first end of the first leg.

10. A stent graft and stent graft delivery system comprising:
a bifurcated stent graft section having including a support coupled to a graft material, said bifurcated stent graft section having a main body and two legs extending distally from said main body, wherein a portion of said support at a distal end of one of said legs has a serpentine shape along a circumference of said distal end which extends beyond an end of the graft material at said distal end such that said serpentine shape forms a plurality of loops at said distal end of said leg;
a delivery system including a helically shaped coil, wherein one or more windings of said coil pass through said loops and prevent disengagement of said loops from said delivery system without relative rotational motion between said coil and said loops at said distal end of said leg of said stent graft section.

11. The stent graft and stent graft delivery system of claim 10, wherein said coil is coupled to a tube extending to a proximal portion of said delivery system.

12. The stent graft and stent graft delivery system of claim 11, further comprising a rotatable element disposed at said proximal portion of said delivery system, wherein said rotatable element is coupled to said tube to rotate said tube and said coil.

13. The stent graft and stent graft delivery system of claim 10, wherein said support includes a plurality of support elements coupled to the graft material.

14. The stent graft and stent graft delivery system of claim 10, wherein the stent graft is self-expandable.

15. A stent graft and stent graft delivery system comprising:
a bifurcated stent graft section having a first end and a second end, said bifurcated stent graft section including graft material and a support coupled to said graft material, wherein a portion of said support at said first end has a serpentine shape along a circumference of said first end which extends beyond an end of the graft material at said first end such that said serpentine shape forms a plurality of loops at said first end of the bifurcated stent graft section; and
a stent graft delivery system, the system comprising:
an elongated inner tube having a proximal end and a tapered, distal end for insertion into a body lumen,
an elongated capture tube having a proximal end, a distal end, a lumen passing there through, and a coil coupled to said distal end, wherein said inner tube is disposed within said lumen of said capture tube such that said capture tube may rotate relative to said inner tube, and
an elongated outer sheath having a proximal end and a distal end and a lumen passing there through, wherein said capture tube and said inner tube are disposed within said lumen of said outer sheath and wherein said bifurcated stent graft section is disposed with said lumen of said outer sheath in a delivery configuration, wherein said outer sheath is slidable relative to said capture tube and said inner tube,
wherein said coil coupled to said capture tube is configured to pass through said plurality of loops of said bifurcated stent graft section to prevent disengagement of said plurality of loops from said capture tube in a first configuration, and wherein said coil is configured to disengage from said plurality of loops upon rotational movement of said capture tube to a second configuration.

16. The stent graft and stent graft delivery system of claim 15, wherein the bifurcated stent graft section includes a main body and two legs extending distally from said main body, wherein said plurality of loops are disposed at a distal end of one of said two legs.

17. The stent graft and stent graft delivery system of claim 15, further comprising a rotatable element disposed at a proximal portion of said delivery system, wherein said rotatable element is coupled to said capture tube to rotate said capture tube and said coil.

18. The stent graft and stent graft delivery system of claim 15, wherein said support includes a plurality of support elements coupled to the graft material.
